(19) United States
(12) Patent Application Publication
(10) Pub. No.: US 2024/0315610 A1
(43) Pub. Date: Sep. 26, 2024

(54) HIGH PERFORMANCE NON-INVASIVE ANALYTE SENSOR.

(71) Applicant: Know Labs, Inc., Seattle, WA (US)

(72) Inventors: Steve LOWE, Seattle, WA (US); Dominic KLYVE, Seattle, WA (US); Steven KENT, Seattle, WA (US)

(21) Appl. No.: 18/585,343

(22) Filed: Feb. 23, 2024

Related U.S. Application Data
(60) Provisional application No. 63/486,833, filed on Feb. 24, 2023.

(51) Int. Cl. A61B 5/145; A61B 5/0507

(52) U.S. Cl. CPC A61B 5/14532; A61B 5/0507; A61B 5/1451; A61B 2562/0228

(57) ABSTRACT

A highly accurate and safe analyte sensor that detects an analyte by transmitting and receiving sensing signals in a radio or microwave frequency range of the electromagnetic spectrum is provided. The analyte sensor has at least two antennas at least one of which operates as a transmit antenna to transmit one or more of the sensing signals and at least one of which operates as a receive antenna, and the analyte sensor meets or exceeds performance parameters for integrated continuous glucose monitoring systems as set forth in 21 C.F.R. 862.1355 (Feb. 18, 2022) and is configured to emit radiofrequency radiation less than radiofrequency radiation exposure limits as set forth in 47 C.F.R. 1.1310 (Apr. 1, 2020).

### FIELD

This disclosure relates generally to apparatus, systems and methods of detecting an analyte via spectroscopic techniques using an analyte sensor that operates in the radio or microwave frequency range of the electromagnetic spectrum.

### BACKGROUND

There is interest in being able to detect and/or measure an analyte within a target. One example is measuring glucose in biological tissue. In the example of measuring glucose in a patient, many current analyte measurement methods are invasive in that they perform the measurement on a bodily fluid such as blood for fingerstick or laboratory-based tests, or on fluid that is drawn from the patient often using an invasive transcutaneous device or using a minimally invasive continuous glucose monitoring device. There are non-invasive methods and devices that claim to be able to perform glucose measurements in biological tissues. However, many of the non-invasive methods and devices generally suffer from: lack of specificity to the analyte of interest, such as glucose; lack of accuracy in determining the analyte of interest; interference from temperature fluctuations; interference from skin compounds (i.e. sweat) and pigments; and complexity of placement, i.e. the sensing device resides on multiple locations on the patient's body. Moreover, the non-invasive methods and devices may not meet the radiofrequency (RF) radiation exposure limits for a user.

### SUMMARY

This disclosure relates generally to apparatus, systems and methods of detecting an analyte via spectroscopic techniques using non-optical frequencies such as in the radio or microwave frequency range of the electromagnetic spectrum. An analyte sensor described herein includes a detector array having a plurality of detector elements (also referred to as antenna elements or antennas) at least one of which can transmit an electromagnetic signal in the radio or microwave frequency range into a target and at least one of which can receive an electromagnetic signal in the radio or microwave frequency range resulting from transmission of the electromagnetic signal. In an embodiment, the detector elements may be part of a common detector array.

The analyte sensor described herein can be used to detect glucose and/or other analyte(s). When used to detect glucose, the sensor can be referred to as a glucose sensor. When used to detect an analyte in general, including glucose and other analytes, the sensor can be referred to as an analyte sensor. The sensor described herein can operate non-invasively whereby the sensor remains completely external to the body from which the glucose or other analyte(s) is sensed. In other embodiments, the sensor can operate in a minimally invasive manner whereby a portion of the sensor pierces the body, the sensor can be invasive whereby the sensor is completely installed in the body, or the sensor can sense glucose or other analyte(s) from a material or fluid that is drawn from and external to the body.

One way to assure the safety or performance of a glucose sensor is by ensuring the glucose sensor meets or exceeds any safety or performance standards as set forth in the Federal Regulations.

In an embodiment, a non-invasive glucose sensor that transmits and receives sensing signals in a radio or microwave frequency range of the electromagnetic spectrum is provided. The glucose sensor has at least two antennas at least one of which operates as a transmit antenna to transmit one or more of the sensing signals and at least one of which operates as a receive antenna, and the glucose sensor meets or exceeds the performance parameters for integrated continuous glucose monitoring systems as set forth in 21 C.F.R. 862.1355 (Feb. 18, 2022).

In one embodiment, a non-invasive glucose sensor that transmits and receives sensing signals in a radio or microwave frequency range of the electromagnetic spectrum is provided. The glucose sensor has at least two antennas at least one of which operates as a transmit antenna to transmit one or more of the sensing signals and at least one of which operates as a receive antenna, and the glucose sensor meets or exceeds at least one or more of the following: 87% or more of the obtained glucose measurements are within ±20% of corresponding actual glucose values of the plurality of adults; when the obtained glucose measurements are less than 70 milligrams/deciliter (mg/dL), 85% or more of the obtained glucose measurements are within ±15 mg/dl of the corresponding actual glucose values of the plurality of adults; when the obtained glucose measurements are between 70 and 180 mg/dL, 70% or more of the obtained glucose measurements are within ±15% of the corresponding actual glucose values of the plurality of adults; when the obtained glucose measurements are greater than 180 mg/dL, 80% or more of the obtained glucose measurements are within ±15% of the corresponding actual glucose values of the plurality of adults; when the obtained glucose measurements are less than 70 mg/dL, 98% or more of the obtained glucose measurements are within ±40 mg/dL of the corresponding actual glucose values of the plurality of adults; when the obtained glucose measurements are between 70 and 180 mg/dL, 99% or more of the obtained glucose measurements are within ±40% of the corresponding actual glucose values of the plurality of adults; when the obtained glucose measurements are greater than 180 mg/dL, 99% or more of the obtained glucose measurements are within ±40% of the corresponding actual glucose values of the plurality of adults; when the obtained glucose measurements are less than 70 mg/dL, the corresponding actual glucose values of the plurality of adults are not greater than 180 mg/dL; when the obtained glucose measurements are greater than 180 mg/dL, the corresponding actual glucose values of the plurality of adults are not less than 70 mg/dL; when the obtained glucose measurements are less than 70 mg/dL, the corresponding actual glucose values of the plurality of adults are not greater than 180 mg/dL; when the corresponding actual glucose values of the plurality of adults is less than-2 mg/dL/minute (/min), no more than 1% of the obtained glucose measurements indicate a positive glucose rate of change greater than 1 mg/dL/min; and when the corresponding actual glucose values of the plurality of adults is greater than 2 mg/dL/min, no more than 1% of the obtained glucose measurements indicate a negative glucose rate of change less than-1 mg/dL/min.

As such, the highly accurate glucose sensor described herein would permit the described glucose sensor to be used that meets the performance parameters for integrated continuous glucose monitoring systems, as set forth in 21 C.F.R. 862.1355, such that the non-invasive glucose sensor can be used as a reference glucose sensor or as a continuous glucose sensor for the monitoring of glucose for a user.

In some embodiments, the glucose sensor simultaneously detects glucose from at least two of blood, interstitial fluid, and cellular material.

In some embodiments, the glucose sensor is a continuous glucose sensor or an on-demand glucose sensor.

In some embodiments, the at least one antenna includes at least two antennas that are part of an antenna array, a transmit circuit that is electrically connectable to at least one transmit antenna and configured to generate the signals to be transmitted by the at least one transmit antenna and a receive circuit that is electrically connectable to at least one receive antenna and configured to receive responses detected by the at least one receive antenna.

In some embodiments, the glucose sensor has from three to six antennas.

In another embodiment, an analyte sensor is described that transmits and receives sensing signals in a radio or microwave frequency range of the electromagnetic spectrum to detect an analyte in a human or animal body. The analyte sensor has at least two antennas at least one of which operates as a transmit antenna to transmit one or more of the sensing signals into the human or animal body and at least one of which operates as a receive antenna, and the analyte sensor is configured to emit RF radiation less than RF radiation exposure limits as set forth in 47 C.F.R. 1.1310 (Apr. 1, 2020).

In yet another embodiment, an analyte sensor is described that transmits and receives sensing signals in a radio or microwave frequency range of the electromagnetic spectrum to detect an analyte in a human or animal body. The analyte sensor has at least two antennas at least one of which operates as a transmit antenna to transmit one or more of the sensing signals into the human or animal body and at least one of which operates as a receive antenna, and the analyte sensor is configured to emit RF radiation according to at least one or more of the following: a specific absorption rate (SAR) of less than 0.4 W/kg over an entire body of a non-user exposed to the glucose sensor; a peak spatial-average of the SAR of less than 8 W/kg to the non-user wearing the glucose sensor averaged over any 1 gram of tissue of the non-user; the SAR of less than 0.08 W/kg for a user of the glucose sensor averaged over an entire body of the user; and the peak spatial average of the SAR of less than 1.6 W/kg for the user of the glucose sensor averaged over any 1 gram of tissue from the entire body of the user.

As such, the highly safe analyte sensor described herein would permit the described analyte sensor to be used that meets the RF radiation exposure limits, for example, telecommunication exposure limits, as set forth in 47 CFR 1.1310, such that the RF radiation exposure has a specific adsorption rate that has low environmental impact on human exposure.

In some embodiments, the analyte sensor is configured to emit the radiofrequency radiation at the peak spatial-average of the SAR at 3.1 W/kg.

In some embodiments, the glucose sensor simultaneously detects glucose from at least two of blood, interstitial fluid, and cellular material.

In some embodiments, the glucose sensor is a continuous glucose sensor or an on-demand glucose sensor.

In some embodiments, the at least one antenna includes at least two antennas that are part of an antenna array, a transmit circuit that is electrically connectable to at least one transmit antenna and configured to generate the signals to be transmitted by the at least one transmit antenna and a receive circuit that is electrically connectable to at least one receive antenna and configured to receive responses detected by the at least one receive antenna.

In some embodiments, the glucose sensor has from three to six antennas.

### BRIEF DESCRIPTION OF DRAWINGS

FIG. 1 is a schematic depiction of an analyte sensor system with an analyte sensor relative to a target according to an embodiment.

FIGS. 2A-2C illustrate different examples of transmit and receive antennas with different geometries.

FIG. 3 illustrates another example of an antenna array that can be used.

FIG. 4 illustrates the penetration depth of RF radiation in body tissue at different RF frequencies.

FIG. 5 is a schematic depiction of a method of using the analyte sensor described herein as a reference or standard glucose sensor against which the performance of other glucose sensors can be compared to determine their accuracy.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with transmitters, receivers, or transceivers and/or medical equipment have not been shown or described in detail to avoid unnecessarily obscuring descriptions of the embodiments.

Unless the context requires otherwise, throughout the specification and claims which follow, the word “comprise” and variations thereof, such as “comprises” and “comprising,” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The following is a detailed description of apparatus, systems and methods of detecting an analyte via spectroscopic techniques using non-optical frequencies such as in the radio or microwave frequency bands of the electromagnetic spectrum. An analyte sensor described herein includes a detector array having a plurality of detector elements (also referred to as antenna elements or antennas) at least one of which can transmit an electromagnetic signal in the radio or microwave frequency range and at least one of which can receive an electromagnetic signal in the radio or microwave frequency range resulting from transmission of the electromagnetic signal. For sake of convenience, the detector array will hereinafter be referred to as an antenna array and the detector elements will hereinafter be referred to as antennas. In an embodiment, the analyte sensor can include a single antenna that functions to both transmit signals and receive returning signals that result from the transmitted signals. In an embodiment, the antennas may be part of a common antenna array. Further information on the construction and operation of analyte sensors are disclosed in U.S. Pat. Nos. 10,548,503; 11,063,373; 11,234,619; 11,031,970; 11,223,383; 11,058,317; 11,058,331; 11,193,923; 11,033,208; 11,529,077; and U.S. Patent Application Publications 2021/0186357; 2021/0244308; 2021/0259571; 2021/0259592; 2021/0259593; 2022/0071523; 2022/0077918; 2022/0077602; 2022/0071527; 2022/0071505; 2022/0074870; 2022/0078471; 2022/0071524; each one of which is incorporated herein by reference in its entirety.

In one embodiment, the analyte sensor described herein can be used to detect the presence of at least one analyte in a target. In another embodiment, the analyte sensor described herein can detect an amount or a concentration of the at least one analyte in the target. The target can be any target containing at least one analyte of interest that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, one or more of blood, interstitial fluid, cerebral spinal fluid, lymph fluid or urine, human tissue, animal tissue, plant tissue, an inanimate object, soil, genetic material, or a microbe.

The analyte(s) can be any analyte that one may wish to detect. The analyte can be human or non-human, animal or non-animal, biological or non-biological. For example, the analyte(s) can include, but is not limited to, one or more of glucose, blood alcohol, oxygen or an indicator thereof, white blood cells, or luteinizing hormone. The analyte(s) can include, but is not limited to, a chemical, a combination of chemicals, a virus, a bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. In an embodiment, the analyte may be simultaneously detected from both blood and interstitial fluid. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-ß hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin and variants thereof including hemoglobin A, hemoglobin S, hemoglobin C, hemoglobin D, hemoglobin E, hemoglobin F, D-Punjab, and beta-thalassemia, particular conformations or conjugations of hemoglobin such as oxyhemoglobin, deoxyhemoglobin, carboxyhemoglobin, and the like; hepatitis B virus; HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free ß-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; zinc protoporphyrin; prostaglandins such as PGF2a and PGE2; hormones such as estrogen, progesterone, and/or follicle stimulating hormone (FSH).

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol or other alcohols; ketones; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

In an embodiment, the analyte(s) are one or more analytes that can be used to determine an oxygen level in a subject. The analytes can be, for example, elemental oxygen, oxyhemoglobin, deoxyhemoglobin, or any other suitable analyte indicative of or a proxy for the oxygen level in the subject. The oxygen level can be an overall level of oxygen or analyte(s) indicative of or a proxy for oxygen by itself, or can be a ratio such as a ratio of oxyhemoglobin to deoxyhemoglobin.

In an embodiment, the analyte(s) can include one or more indicators for determination of hydration of a subject. The analyte(s) can include, for example, hemoglobin, red blood cells as a whole, one or more hormones, sodium, one or more solutes from which osmolarity can be determined, or the like. The amount of the analyte(s) can be used to determine one or more indicia of hydration, such as concentrations of one or more analytes, hematocrit, osmolarity, or any other suitable measurement of a hydration level of the subject. The osmolarity can be an osmolarity of one or more of plasma, interstitial fluid, saliva, urine, or the like. In an embodiment, a sensor can be positioned such that the results of detection are indicative of the presence or amount of analytes in the bladder of the subject, such that urine parameters related to hydration such as urine osmolarity can be determined. In an embodiment, the sensor can be positioned such that results of detection are indicative of the presence or amount of analytes in saliva. A hydration level can be determined based on the one or more indicators, for example by comparing osmolarity or hematocrit to reference values. The reference values can be reference values specific to the subject, general reference values, reference values for a group that the subject belongs to, or the like. In an embodiment, the sensor can detect the one or more analytes in the subject non-invasively. In an embodiment, the sensor can detect the one or more analytes in a sample obtained from the subject, such as a blood, urine, or saliva sample. The sample can have a predetermined mass or volume.

In an embodiment, the sensor described herein can be incorporated into a wearable device such as a ring, a watch, or any other suitable wearable device that is worn on the user's body. The wearable device may be configured to be worn by the user over a longer period of time, for example a watch, a ring, or the like. Alternatively, the wearable device may be configured to be temporarily worn, for example only during one or more analyte readings after which the wearable device is removed. In an embodiment, the sensor described herein can be configured as a non-wearable device. For example, the sensor can be configured as a device that a user holds or presses against a body part during an analyte reading, or a body part is pressed against the sensor, during an analyte reading.

The device including the sensor, whether wearable or non-wearable, can also be configured to be capable of detecting one or more physiological parameters such as user heart rate, user blood pressure, user body temperature, user calorie consumption, user glucose level, user sweat production, one or more hormone levels, bioelectric impedance, or the like. One or more of the physiological parameters can be detected directly using the sensor and/or determined based on detection of one or more analytes by the sensor. In an embodiment, one or more of the physiological parameters can be detected or determined using one or more additional physiological sensors included in the device in addition to the sensor described herein. The one or more additional physiological sensors can be any suitable physiological sensor for the particular physiological parameter to be sensed. In an embodiment, one or more of the physiological parameters can be determined based on a presence or amount of one or more analytes detected by the sensor and one or more additional measurements made by one or more additional physiological sensors included in the device. The device can also include one or more additional functionalities including, but not limited to, a camera; an accelerometer; a pedometer; a fitness/activity tracker; an altimeter; a barometer; a compass; a global positioning system receiver; a sleep monitor; a fall sensor; a microphone; a speaker; and others.

The analyte sensor described herein can be used to detect glucose and/or other analyte(s). When used to detect glucose, the sensor can be referred to as a glucose sensor. When used to detect an analyte in general, including glucose and other analytes, the sensor can be referred to as an analyte sensor. The sensor described herein can operate non-invasively whereby the sensor remains completely external to the body from which the glucose or other analyte(s) is sensed and the detection of the glucose or other analyte occurs without requiring removal of fluid or other removal from the target, such as the human body. In other embodiments, the sensor can operate in a minimally invasive manner whereby a portion of the sensor pierces the body, the sensor can be invasive whereby the sensor is completely installed in the body, or the sensor can sense glucose or other analyte(s) from a material or fluid that is drawn from and external to the body.

The analyte sensors described herein operate by transmitting an electromagnetic signal in the radio or microwave frequency range of the electromagnetic spectrum toward and into a target using a transmit antenna. A returning signal that results from the transmission of the transmitted signal is detected by a receive antenna. The signal(s) detected by the receive antenna can be analyzed to detect the analyte based on the intensity of the received signal(s) and reductions or increases in intensity at one or more frequencies where the analyte absorbs the transmitted signal and/or reflection or refraction of the signal, e.g., the measured value at a given frequency point can go up or down depending on which side of a resonant minima they are as the resonance shifts. An example of detecting an analyte using a non-invasive spectroscopy sensor operating in the radio or microwave frequency range of the electromagnetic spectrum is described in U.S. Pat. No. 10,548,503, the entire contents of which are incorporated herein by reference. The signal(s) detected by the receive antenna can be complex signals including a plurality of signal components, each signal component being at a different frequency. In an embodiment, the detected complex signals can be decomposed into the signal components at each of the different frequencies, for example through a Fourier transformation. In an embodiment, the complex signal detected by the receive antenna can be analyzed as a whole (i.e., without demultiplexing the complex signal) to detect the analyte as long as the detected signal provides enough information to make the analyte detection. In addition, the signal(s) detected by the receive antenna can be separate signal portions, each having a discrete frequency.

The analyte sensor system, for example a processor-based system, can also be operable to determine differences between the response signals and the respective excitation signals that gave rise to the respective response signals. Thus, the processor-based system is operable to assess signals such as S parameters and/or transition line parameters and/or dielectric parameters. An example being the amount of gain or loss (e.g., dB) between the excitation signals and the corresponding response signals which results from passage of the signals through at least a portion of bodily tissue that is being assessed or sampled, e.g., a target. At least some of these determined differences are the result of, and hence characterize, one or more physical conditions or states of the bodily tissue or concentrations of material within the bodily tissue at the time of the assessment or performance of the medical diagnostics, referred to herein as sampling.

The analyte sensor system can also be configured to compare the determined differences to a set of baseline determined differences, collected at a previous time, and which characterize one or more physical conditions or states of the bodily tissue at a baseline state, for example producing a set of differences between the current state values and the baseline state values. The baseline state may represent a healthy state, or may simply represent a starting state, whether the subject is considered healthy at that time or not. The baseline state may represent a baseline for the particular subject being assessed, or may represent a generic baseline common across many subjects. The analyte sensor system may compare a difference between one or more of the sampling state values (e.g., differences between the excitation and response signals captured at a current or sampling time) and one or more of the baseline state values (e.g., differences between the excitation and response signals captured at a baseline time). The analyte sensor system may assess whether a defined pattern exists or is absent from the differences, and provide an indication of a presence or absence of an anomalous physical condition or other difference or null difference of the subject based on the comparison identified.

In an embodiment, the analyte sensor can include a trained machine-learning model for analyzing the signals discussed above. The trained machine-learning model can be based on a neural network or convolutional neural network that includes regression models to provide a model or algorithm for analyzing the signals. In an embodiment, the machine-learning model can be trained on data collected from an another or reference analyte sensor that can be an invasive, minimally invasive, or non-invasive analyte sensor. The machine-learning model can then be established using the training data and the data collected by the another or reference sensor by comparing various neural network architectures and hyperparameter settings, in which neurons or layers are trained to predict the training data using the hyperparameters and various models. The neural network architecture that provides the best performance and/or smallest validation error can be chosen as the trained machine-learning model, which can be deployed for inference, e.g., downloaded on a processor-enabled device or accessed on a server or cloud-based server.

For sake of convenience, hereinafter the sensor will be described as sensing glucose and the sensor will be referred to as a glucose sensor. When used non-invasively, the glucose sensor described herein simultaneously obtains glucose readings from blood, interstitial fluid and cellular material which increases the accuracy of the glucose sensor compared to conventional glucose sensors, such as minimally invasive continuous glucose monitors (CGMs) and fingerstick sensors, which obtain glucose readings from one source, namely interstitial fluid in the case of minimally invasive CGMs and blood in the case of fingerstick sensors. However, in one embodiment, the glucose sensor described herein can be used to detect glucose from just blood, for example blood that has been drawn from a body; or from just interstitial fluid; from just cellular material; or from any two of these materials.

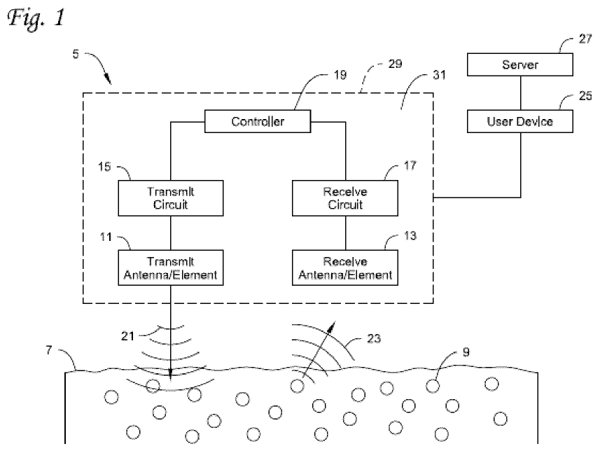

FIG. 1 illustrates the glucose sensor configured as a non-invasive glucose sensor that uses two or more antennas including one that functions as a transmit antenna and one that functions as a receive antenna. The transmit antenna and the receive antenna can be located near the target and operated as further described herein to assist in detecting glucose in the target. The transmit antenna transmits a signal toward and into the target. The receive antenna detects a response resulting from transmission of the signal by the transmit antenna into the target.

The transmit antenna and the receive antenna can be decoupled (which may also be referred to as detuned or the like) from one another. Decoupling refers to intentionally fabricating the configuration and/or arrangement of the transmit antenna and the receive antenna to minimize direct communication between the transmit antenna and the receive antenna, preferably absent shielding. Shielding between the transmit antenna and the receive antenna can be utilized. However, the transmit antenna and the receive antenna are decoupled even without the presence of shielding.

Referring now to FIG. 1, an embodiment of a non-invasive glucose sensor system with a non-invasive glucose sensor 5 is illustrated. The sensor 5 is depicted relative to a target 7 from which the glucose 9 is to be detected. The glucose 9 can be in blood of the target 7, interstitial fluid of the target 7, and cellular material of the target 7. In this example, the sensor 5 is depicted as including an antenna array that includes a transmit antenna/element 11 (hereinafter “transmit antenna 11”) and a receive antenna/element 13 (hereinafter “receive antenna 13”). The sensor 5 further includes a transmit circuit 15, a receive circuit 17, and a controller 19. The sensor 5 can also include a power supply, such as a battery (not shown in FIG. 1). In some embodiments, power can be provided from mains power, for example by plugging the sensor 5 into a wall socket via a cord connected to the sensor 5.

The transmit antenna 11 is positioned, arranged and configured to transmit a signal 21 that is in the radio frequency (RF) or microwave range of the electromagnetic spectrum into the target 7. The transmit antenna 11 can be an electrode or any other suitable transmitter of electromagnetic signals in the radio frequency (RF) or microwave range. The transmit antenna 11 can have any arrangement and orientation relative to the target 7 that is sufficient to allow the analyte sensing to take place. In one non-limiting embodiment, the transmit antenna 11 can be arranged to face in a direction that is substantially toward the target 7.

The signal 21 transmitted by the transmit antenna 11 is generated by the transmit circuit 15 which is electrically connectable to the transmit antenna 11. The transmit circuit 15 can have any configuration that is suitable to generate a transmit signal to be transmitted by the transmit antenna 11. Transmit circuits for generating transmit signals in the RF or microwave frequency range are well known in the art. In one embodiment, the transmit circuit 15 can include, for example, a connection to a power source, a frequency generator, and optionally filters, amplifiers or any other suitable elements for a circuit generating an RF or microwave frequency electromagnetic signal. In an embodiment, the signal generated by the transmit circuit 15 can have frequency that is in the range from about 10 kHz to about 100 GHz. In another embodiment, the frequency can be in a range from about 300 MHz to about 6000 MHz. In an embodiment, the transmit circuit 15 can be configured to sweep through a range of frequencies that are within the range of about 10 kHz to about 100 GHz, or in another embodiment a range of about 500 MHz to about 3000 MHz at 1 MHz intervals. In an embodiment, the transmit circuit 15 can be configured to perform each sweep over a 6 second period, e.g., a dwell time at each frequency of about 50 milliseconds, and can include a one second pause between sweeps. In an embodiment, the transmission power, e.g., when measured at the antenna feedpoint, can be between 10 and 20 mW, and preferably about 16 mW, e.g., ±5%. In an embodiment, the transmission power may be the maximum available power from the sensor to generate the signals. In order to reduce the noise of the signal, in an embodiment, the transmission power can be averaged at each frequency of the sweep, e.g., to provide a single power measurement per dwell.

The receive antenna 13 is positioned, arranged, and configured to detect one or more electromagnetic response signals 23 that result from the transmission of the transmit signal 21 by the transmit antenna 11 into the target 7 and impinging on the glucose molecules 9. The receive antenna 13 can be an electrode or any other suitable receiver of electromagnetic signals in the radio frequency (RF) or microwave range. In an embodiment, the receive antenna 13 is configured to detect electromagnetic signals having a frequency that is in the range from about 10 kHz to about 100 GHz, or in another embodiment a range from about 300 MHz to about 6000 MHz, and preferably between 500 MHz and 3000 MHz or between 500 MHz and 2500 MHz. The receive antenna 13 can have any arrangement and orientation relative to the target 7 that is sufficient to allow detection of the response signal(s) 23 to allow the glucose sensing to take place. In one non-limiting embodiment, the receive antenna 13 can be arranged to face in a direction that is substantially toward the target 7.

The receive circuit 17 is electrically connectable to the receive antenna 13 and conveys the received response from the receive antenna 13 to the controller 19. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19.

The controller 19 controls the operation of the sensor 5. The controller 19, for example, can direct the transmit circuit 15 to generate a transmit signal to be transmitted by the transmit antenna 11. The controller 19 further receives signals from the receive circuit 17. The controller 19 can optionally process the signals from the receive circuit 17 to detect the analyte(s), e.g., glucose, 9 in the target 7. In one embodiment, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to detect the glucose molecules 9. If provided, the external device 25 may be used to provide communication between the sensor 5 and the remote server 27, for example using a wired data connection or via a wireless data connection or Wi-Fi of the external device 25 to provide the connection to the remote server 27. In an embodiment, the controller 19 and/or the remove server 27 may include a processor or microprocessor for executing a trained machine-learning model that is configured to process the signals to detect glucose.

In an embodiment, the controller 19 can process the signals from the receive circuit 17 to detect the analyte(s) 9, e.g., glucose, in the target 7 using the trained machine-learning model. The trained machine-learning model can be based on a neural network or convolutional neural network that includes regression models to provide a model or algorithm for analyzing the signals. In an embodiment, the machine-learning model can be trained on data collected from an another or reference analyte sensor that can be an invasive, minimally invasive, or non-invasive analyte sensor.

For example, in an embodiment, the machine-learning model for the glucose sensor can be trained on data collected from a minimally invasive continuous glucose monitoring device, such as Dexcom® G6. The minimally invasive continuous glucose monitoring device can be used to obtain the training data by measuring the glucose level of a subject after the subject consumes controlled amounts of a glucose containing substance, such as, liquid D-glucose. The minimally invasive continuous glucose monitoring device can also be used to obtain data after the subject did not consume the D-glucose. Throughout the obtaining of the training data, data can be collected on a continuous basis using the glucose sensor by obtaining signals of the frequency during sweeps across the 500 MHZ-3000 MHz range at 1 MHz intervals. The full sweep of the frequencies can take approximately 7 seconds which can include at least a one second pause between sweeps. As such, over a 2-5 hour test, and preferably a 3 hour test, between 1000 and 2600 sweeps can be performed to result in approximately 2.5 to 6.5 million pieces of data being collected. The signals can be smoothed using a filter, such as a Savitzky-Golay filter with a window length of 2000, polynomial order of 4, derivative order of 1 and extension mode “nearest.” The machine-learning model can then be trained using the large number of training data and the data collected by the glucose sensor to establish a robust model by comparing various neural network architectures and hyperparameter settings, e.g., neurons or layers trained to predict the training data. The neural network architecture that provides the best performance and/or smallest validation error can then be chosen as the trained machine-learning model, which can be deployed for inference on the processor-enabled controller 19. A machine-learning model that has been trained on such a robust data collection scheme has not been previously attainable.

With continued reference to FIG. 1, the sensor 5 may include a sensor housing 29 (shown in dashed lines) that defines an interior space 31. Components of the sensor 5 may be attached to and/or disposed within the housing 29. For example, the transmit antenna 11 and the receive antenna 13 are attached to the housing 29. In some embodiments, the antennas 11, 13 may be entirely or partially within the interior space 31 of the housing 29. In some embodiments, the antennas 11, 13 may be attached to the housing 29 but at least partially or fully located outside the interior space 31. In some embodiments, the transmit circuit 15, the receive circuit 17 and the controller 19 are attached to the housing 29 and disposed entirely within the sensor housing 29. In some embodiments, the antennas 11, 13 can be from 0.5 to 5 mm from a surface of the target 7, e.g., the skin surface, and preferably about 1 mm from the surface of the target 7.

The receive antenna 13 may be decoupled or detuned with respect to the transmit antenna 11 such that electromagnetic coupling between the transmit antenna 11 and the receive antenna 13 is reduced. The decoupling of the transmit antenna 11 and the receive antenna 13 increases the portion of the signal(s) detected by the receive antenna 13 that is the response signal(s) 23 from the target 7, and minimizes direct receipt of the transmitted signal 21 by the receive antenna 13.

In an embodiment, coupling between the transmit antenna 11 and the receive antenna 13 is 95% or less. In another embodiment, coupling between the transmit antenna 11 and the receive antenna 13 is 90% or less. In another embodiment, coupling between the transmit antenna 11 and the receive antenna 13 is 85% or less. In another embodiment, coupling between the transmit antenna 11 and the receive antenna 13 is 75% or less.

Any technique for reducing coupling between the transmit antenna 11 and the receive antenna 13 can be used. For example, the decoupling between the transmit antenna 11 and the receive antenna 13 can be achieved by one or more intentionally fabricated configurations and/or arrangements between the transmit antenna 11 and the receive antenna 13 that is sufficient to decouple the transmit antenna 11 and the receive antenna 13 from one another.

For example, in one embodiment described further below, the decoupling of the transmit antenna 11 and the receive antenna 13 can be achieved by intentionally configuring the transmit antenna 11 and the receive antenna 13 to have different geometries from one another. Intentionally different geometries refers to different geometric configurations of the transmit and receive antennas 11, 13 that are intentional. Intentional differences in geometry are distinct from differences in geometry of transmit and receive antennas that may occur by accident or unintentionally, for example due to manufacturing errors or tolerances.

Another technique to achieve decoupling of the transmit antenna 11 and the receive antenna 13 is to provide appropriate spacing between each antenna 11, 13 that is sufficient to decouple the antennas 11, 13 and force a proportion of the electromagnetic lines of force of the transmitted signal 21 into the target 7 thereby minimizing or eliminating as much as possible direct receipt of electromagnetic energy by the receive antenna 13 directly from the transmit antenna 11 without traveling into the target 7. The appropriate spacing between each antenna 11, 13 can be determined based upon factors that include, but are not limited to, the output power of the signal from the transmit antenna 11, the size of the antennas 11, 13, the frequency or frequencies of the transmitted signal, and the presence of any shielding between the antennas. This technique helps to ensure that the response detected by the receive antenna 13 is measuring the glucose molecules 9 and is not just the transmitted signal 21 flowing directly from the transmit antenna 11 to the receive antenna 13. In some embodiments, the appropriate spacing between the antennas 11, 13 can be used together with the intentional difference in geometries of the antennas 11, 13 to achieve decoupling.

In one embodiment, the transmit signal (or each of the transmit signals) can be transmitted over a transmit time or dwell time that is less than, equal to, or greater than about 300 ms. In another embodiment, the transmit or dwell time can be less than, equal to, or greater than about 200 ms. In still another embodiment, the transmit or dwell time can be less than, equal to, or greater than about 30 ms or greater than or equal to about 50 ms. The transmit or dwell time could also have a magnitude that is measured in seconds, for example 1 second, 5 seconds, 10 seconds, or more. In an embodiment, the same transmit signal can be transmitted multiple times, and then the transmit time can be averaged. In another embodiment, the transmit signal (or each of the transmit signals) can be transmitted with a duty cycle that is less than or equal to about 50%.

As mentioned above, one technique for decoupling the transmit antenna 11 from the receive antenna 13 is to intentionally configure the transmit antenna 11 and the receive antenna 13 to have intentionally different geometries. Intentionally different geometries refer to differences in geometric configurations of the transmit and receive antennas 11, 13 that are intentional, and is distinct from differences in geometry of the transmit and receive antennas 11, 13 that may occur by accident or unintentionally, for example due to manufacturing errors or tolerances when fabricating the antennas 11, 13.

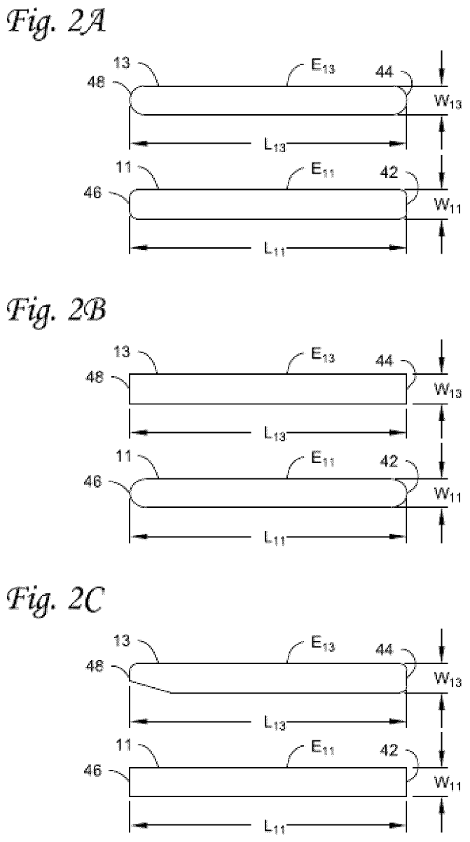

The different geometries of the antennas 11, 13 may manifest itself, and may be described, in a number of different ways. For example, in a plan view of each of the antennas 11, 13 (such as in FIGS. 2A-C), the shapes of the perimeter edges of the antennas 11, 13 may be different from one another. The different geometries may result in the antennas 11, 13 having different surface areas in plan view. The different geometries may result in the antennas 11, 13 having different aspect ratios in plan view (i.e. a ratio of their sizes in different dimensions; for example, as discussed in further detail below, the ratio of the length divided by the width of the antenna 11 may be different than the ratio of the length divided by the width for the antenna 13). In some embodiments, the different geometries may result in the antennas 11, 13 having any combination of different perimeter edge shapes in plan view, different surface areas in plan view, and/or different aspect ratios.

So as used herein, a difference in geometry or a difference in geometrical shape of the antennas 11, 13 refers to any intentional difference in the figure, length, width, size, shape, arca closed by a boundary (i.e. the perimeter edge), etc. when the respective antenna 11, 13 is viewed in a plan view.

The antennas 11, 13 can have any configuration and can be formed from any suitable material that allows them to perform the functions of the antennas 11, 13 as described herein. In one embodiment, the antennas 11, 13 can be formed by strips of material. A strip of material can include a configuration where the strip has at least one lateral dimension thereof greater than a thickness dimension thereof when the antenna is viewed in a plan view (in other words, the strip is relatively flat or of relatively small thickness compared to at least one other lateral dimension, such as length or width when the antenna is viewed in a plan view as in FIGS. 2A-C). A strip of material can include a wire. The antennas 11, 13 can be formed from any suitable conductive material(s) including metals and conductive non-metallic materials. Examples of metals that can be used include, but are not limited to, copper or gold. Another example of a material that can be used is non-metallic materials that are doped with metallic material to make the non-metallic material conductive.

FIG. 2A illustrates a plan view of an antenna array having two antennas with different geometries. In this example, the antennas 11, 13 are illustrated as substantially linear strips. In this example, the antennas 11, 13 differ in geometry from one another in that the shapes of the ends of the antennas 11, 13 differ from one another. For example, when viewing FIG. 2A, the right end 42 of the antenna 11 has a different shape than the right end 44 of the antenna 13. Similarly, the left end 46 of the antenna 11 may have a similar shape as the right end 42, but differs from the left end 48 of the antenna 13 which may have a similar shape as the right end 44. It is also possible that the lateral lengths L₁₁, L₁₃ and/or the lateral widths W₁₁, W₁₃ of the antennas 11, 13 could differ from one another.

FIG. 2B illustrates another plan view of an antenna array having two antennas with different geometries that is somewhat similar to FIG. 2A. In this example, the antennas 11, 13 are illustrated as substantially linear strips, and the antennas 11, 13 differ in geometry from one another in that the shapes of the ends of the antennas 11, 13 differ from one another. For example, when viewing FIG. 2B, the right end 42 of the antenna 11 has a different shape than the right end 44 of the antenna 13. Similarly, the left end 46 of the antenna 11 may have a similar shape as the right end 42, but differs from the left end 48 of the antenna 13 which may have a similar shape as the right end 44. In addition, the lateral widths W₁₁, W₁₃ of the antennas 11, 13 differ from one another. It is also possible that the lateral lengths L₁₁, L₁₃ of the antennas 11, 13 could differ from one another.

FIG. 2C illustrates another plan view of an antenna array having two antennas with different geometries that is somewhat similar to FIGS. 2A and 2B. In this example, the antennas 11, 13 are illustrated as substantially linear strips, and the antennas 11, 13 differ in geometry from one another in that the shapes of the ends of the antennas 11, 13 differ from one another. For example, when viewing FIG. 2C, the right end 42 of the antenna 11 has a different shape than the right end 44 of the antenna 13. Similarly, the left end 46 of the antenna 11 may have a similar shape as the right end 42, but differs from the left end 48 of the antenna 13 which may have a similar shape as the right end 44. In addition, the lateral widths W₁₁, W₁₃ of the antennas 11, 13 differ from one another. It is also possible that the lateral lengths L₁₁, L₁₃ of the antennas 11, 13 could differ from one another.

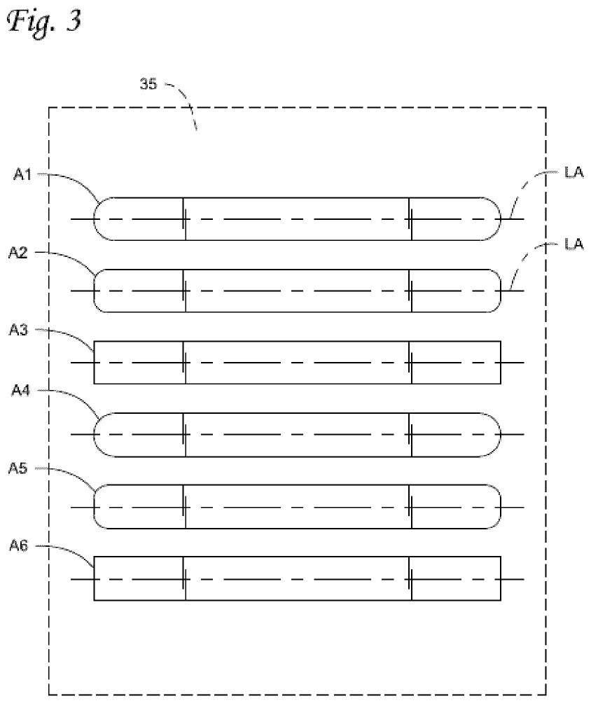

The examples in FIGS. 1 and 2A-C depict the use of two antennas. However, a different number of antennas can be used, as long as at least one of the antennas can function as a transmit antenna and as long as at least one of the antennas can function as a receive antenna. FIG. 3 illustrates a plan view of an antenna array having six antennas illustrated as substantially linear strips. In this example, the antennas differ in geometry from one another in that the shapes of the ends of the antennas, the lateral lengths and/or the lateral widths of the antennas may differ from one another. However, in one embodiment three antennas can be used, four antennas can be used, five antennas can be used, etc.

Referring to FIG. 3, the antennas are labeled in order A1-A6. Each antenna A1-A6 can function as either a transmit antenna or as a receive antenna. In another embodiment, each antenna A1-A6 can operate solely as a transmit antenna or as a receive antenna. All of the antennas A1-A6 are depicted as being disposed on the same substrate 35. However, the antennas A1-A6 can be disposed on two or more substrates. In addition, each one of the antennas A1-A6 has a longitudinal axis LA (depicted in dashed lines), and the longitudinal axes LA of the antennas A1-A6 are illustrated as being parallel to each other. However, the longitudinal axes LA need not be parallel. Some of the longitudinal axes may be parallel to one another while others are angled; or all of the longitudinal axes may be angled (i.e., not parallel to one another).

Further, at least one of the antennas A1-A6 has a rectangular shape, at least one of the antennas A1-A6 has a stadium shape, and at least one of the antennas A1-A6 has a rounded rectangle shape. In the illustrated embodiment, two of the antennas, such as the antennas A3 and A6, have a rectangular shape; two of the antennas, such as the antennas A1 and A4, have a stadium shape; and two of the antennas, such as the antennas A2 and A5, have a rounded rectangle shape. A stadium shape is a two-dimensional geometric shape constructed of a rectangle with semicircles at opposite ends. A rounded rectangle shape is a two-dimensional geometric shape constructed of a rectangle with radiuses at each corner of the rectangle. The antennas in FIG. 3 are arranged on the substrate 35 in a manner such that two antennas with the same shape are not located next to one another.

The antenna array in FIG. 3 is configured such that a first one of the antennas, such as the antenna A1 or A4, has a first perimeter length; a second one of the antennas, such as the antenna A2 or A5, has a second perimeter length; and a third one of the antennas, such as the antenna A3 or A6, has a third perimeter length. In one embodiment, the first perimeter length, the second perimeter length, and the third perimeter length differ from one another. The difference in perimeter lengths is due to the different geometric shapes of the antennas. In another embodiment, the antennas A1-A6 can have different geometric shapes but some or all of the perimeter lengths of the antennas A1-A6 can be the same. In still another embodiment, the antennas A1-A6 can have the same geometric shape but different perimeter lengths. In one embodiment, each antenna A1-A6 can have the same maximum longitudinal length L and the same maximum width W, with the different geometrical shapes accounting for the different perimeter lengths.

At least based on the foregoing structures and functions, the glucose sensor described herein is highly accurate. One way to assess the accuracy of a glucose sensor is ensuring that the glucose sensor meets or exceeds the performance parameters for integrated continuous glucose monitoring systems as set forth in 21 C.F.R. 862.1355 (Feb. 18, 2022), which is incorporated herein by reference. Pursuant to parameters set forth in 21 C.F.R. 862.1355, the glucose sensor as described herein meets one or more of the controls for verification and validation based on a comparison of the glucose sensor values and blood glucose values in specimens collected in parallel that are measured on an FDA-accepted laboratory-based glucose measurement method that is precise and accurate (actual glucose values from a reference glucose sensor), and that is traceable to a higher order (e.g., an internationally recognized reference material and/or method) as follows:

For the glucose sensor measurements less than 70 milligrams/deciliter (mg/dL), 85% or more of the obtained glucose measurements are within ±15 mg/dL of the corresponding actual glucose values of the plurality of adults.

For the glucose sensor measurements between 70 and 180 mg/dL, 70% or more of the obtained glucose measurements are within ±15% of the corresponding actual glucose values of the plurality of adults.

For the glucose sensor measurements greater than 180 mg/dL, 80% or more of the obtained glucose measurements are within ±15% of the corresponding actual glucose values of the plurality of adults.

For the glucose sensor measurements less than 70 mg/dL, 98% or more of the obtained glucose measurements are within ±40 mg/dL of the corresponding actual glucose values of the plurality of adults.

For the glucose sensor measurements between 70 and 180 mg/dL, 99% or more of the obtained glucose measurements are within ±40% of the corresponding actual glucose values of the plurality of adults.

For the glucose sensor measurements greater than 180 mg/dL, 99% or more of the obtained glucose measurements are within ±40% of the corresponding actual glucose values of the plurality of adults.

For the glucose sensor measurements, 87% or more of the obtained glucose measurements are within ±20% of corresponding actual glucose values of the plurality of adults.

For the glucose sensor measurements less than 70 mg/dL, the corresponding actual glucose values of the plurality of adults is not greater than 180 mg/dL.

For the glucose sensor measurements greater than 180 mg/dL, the corresponding actual glucose values of the plurality of adults is not less than 70 mg/dL.

For the glucose measurements less than 70 mg/dL, the corresponding actual glucose values of the plurality of adults is not greater than 180 mg/dL.

For corresponding actual glucose values of the plurality of adults of less than-2 mg/dL/minute (/min), no more than 1% of the glucose sensor measurements indicate a positive glucose rate of change greater than 1 mg/dL/min.

For the corresponding actual glucose values of the plurality of adults is greater than 2 mg/dL/min, no more than 1% of the glucose sensor measurements indicate a negative glucose rate of change less than-1 mg/dL/min.

The glucose sensor measurement described herein can thus be based on a comparison between one or more glucose readings obtained by the glucose sensor described herein with one or more glucose readings contemporaneously obtained by the FDA-accepted laboratory-based glucose measurement method that is precise and accurate, e.g., a reference glucose device that can be an invasive or minimally invasive continuous glucose sensor. The plurality of adults that are tested can be a group of adults between 5 and 1000, or greater, who are tested over a period of time that exceeds 1 day, e.g., 24 hours, 48 hours, 7 days, a month, etc. As such, the glucose sensor described herein, and specifically, a non-invasive glucose sensor described herein, has an accuracy that is greater than an accuracy of non-invasive glucose sensors, and has an accuracy that is the same as or greater than the accuracy of commercially available glucose sensors such as minimally invasive continuous glucose sensor and fingerstick glucose sensor. Examples of minimally invasive continuous glucose sensors are, but are not limited to, the FreeStyle Libre® CGM, the Dexcom® G6, and many others. An example of a fingerstick glucose sensor includes, but is not limited to, a OneTouch® Ultra®2.

There may be a number of reasons why the non-invasive glucose sensor described herein has a high accuracy. One reason for the high accuracy of the glucose sensor described herein is believed to be that the sensor simultaneously obtains glucose readings from two or more of blood, interstitial fluid and cellular material. Conventional glucose sensors, such as minimally invasive continuous glucose monitors (CGMs) and fingerstick sensors, obtain glucose readings from one source, namely interstitial fluid in the case of minimally invasive CGMs and blood in the case of fingerstick sensors. Accordingly, the glucose sensor transmits and receives sensing signals in a radio or microwave frequency range of the electromagnetic spectrum at specific transmission powers, with the glucose sensor having at least two antennas at least one of which operates as a transmit antenna to transmit one or more of the sensing signals and at least one of which operates as a receive antenna, and the glucose sensor simultaneously detects glucose from blood, interstitial fluid, and cellular material.

Another reason for the high accuracy of the glucose sensor described herein is believed to be the trained machine-learning model used to process the signals to detect the glucose. As discussed above, since the training data is robust in size, e.g., having over 1 million pieces of collected data, the machine-learning model can be trained more accurately based on the sensed signals and training data. As such, the trained machine-learning model may include hyperparameters and a regression-based neural network that accurately and precisely predicts the glucose reading from the received signals. In an embodiment, the signal(s) can be analyzed to detect the glucose based on the intensity of the received signal(s) and/or reductions in intensity at one or more frequencies where the glucose absorbs the transmitted signal and/or reflection or refraction of the signal. In an embodiment, the signal(s) can be analyzed to determine differences between the response signals and the respective excitation signals that gave rise to the respective response signals. Thus, the processor-based glucose sensor is operable to assess signals such as S parameters and/or transition line parameters and/or dielectric parameters which can correspond to hyperparameters in the machine-learning model. In another embodiment, the glucose sensor can also be configured to compare the determined differences to a set of baseline determined differences, collected at a previous time, and which characterize one or more physical conditions or states of the bodily tissue at a baseline state, for example producing a set of differences between the current state values and the baseline state values. The baseline state may represent a healthy state, or may simply represent a starting state, whether the subject is considered healthy at that time or not. The baseline state may represent a baseline for the particular subject being assessed, or may represent a generic baseline common across many subjects.

Additional reasons for the high accuracy of the glucose sensor described herein are believed to be, but are not necessarily limited to, the control of frequency sweeps as described in U.S. Pat. No. 11,033,208, the entire contents of which are incorporated herein by reference in their entirety; the ability to use different combinations of transmit and receive antennas as described in U.S. Pat. No. 11,058,331, the entire contents of which are incorporated herein by reference in their entirety; and the use of different antenna geometries as described herein.

Additionally, based on the foregoing structures and functions, the analyte sensor described herein is highly safe. One way to assess the safety of the analyte sensor is to determine an amount of radiation absorbed by the user's body and ensuring that the analyte sensor emits RF radiation that is below the exposure limits, e.g., safe limits, for other devices, such as telecommunications equipment, as set forth in 47 C.F.R. 1.310 (Apr. 1, 2020), which is incorporated herein by reference. Pursuant to parameters set forth in 47 C.F.R. 1.310, the analyte sensor as described herein meets one or more of the specific absorption rate (SAR) limits which are used to evaluate the environmental impact of human exposure to RF radiation within the frequency range of 10 kHz to 6 GHz as follows:

A SAR limit for occupational/controlled exposure of less than 0.4 W/kg, as averaged over the whole body, and a peak spatial-average SAR of less than 8 W/kg, averaged over any 1 gram of tissue (defined as a tissue volume in the shape of a cube). Exposure may be averaged over a time period not to exceed 6 minutes to determine compliance with occupational/controlled SAR limits. The occupational/controlled exposure limits apply in situations in which persons are exposed as a consequence of their employment and can exercise control over their exposure, e.g., a non-user of the analyte sensor who may be exposed due to proximity of the analyte sensor.

A SAR limit for general population/uncontrolled exposure of less than 0.08 W/kg, as averaged over the whole body, and a peak spatial-average SAR of 1.6 W/kg, averaged over any 1 gram of tissue (defined as a tissue volume in the shape of a cube). Exposure may be averaged over a time period not to exceed 30 minutes to determine compliance with general population/uncontrolled SAR limits. The general population/uncontrolled exposure limits apply in situations in which the general public may be exposed, for example, RF sources intended for consumer use, e.g., a user of the analyte sensor.

In an embodiment, an analyte sensor as described herein can be configured to meet such safety parameters. For example, the sensor can include an antenna array that includes a transmit antenna/element (hereinafter “transmit antenna”) and a receive antenna/element (hereinafter “receive antenna”) that have design topologies for narrow and broad-band radiating structures, e.g., loops, monopoles, patches, spirals, etc. The antenna array can have a size between 1×1 mm and 250×250 mm, and preferably a size of 30×30 mm. The antenna elements can be designed such that the elements do not radiate efficiently into free space or for any specific resonant frequency, but rather provide a mechanism for RF fields to be capacitively coupled, e.g., between the transmit antenna and the receive antenna. The sensor can further include a transmit circuit, a receive circuit, and a controller or microcontroller. The sensor can also include a printed circuit board assembly (PCBA), such as described in U.S. Patent Publication Number 2023/0145527, filed Jun. 3, 2022, which is incorporated by reference. The PCBA can include the transmit antenna, the receive antenna, transmit circuit, receive circuit, and controller or microcontroller to generate the RF signals and to measure received power after passing the signals through the antenna array. The RF signals can range from 100 MHz to 4 GHZ, and preferably ranges from 500 MHz to 3000 MHz or from 500 MHz to 2500 MHz. The PCBA can also include a transmit amplifier to boost the signal and a switch matrix for sending the signal to any one of the antenna elements, or through an onboard fixed-attenuation path which allows the system to test itself and provide a known benchmark. The switch matrix can be used to establish the receive path, in which one of the supported antenna elements is chosen to receive the transmitted signal. A low noise amplifier can be used to amplify the signal for measurement by a power measurement circuit, e.g., a log amp, which is configured to translate the received RF signal's power into a voltage output that can be sampled by an analog to digital converter.

The safety of the analyte sensor, as determined by the RF radiation emitted by the sensor, can be determined as follows. In determining the amount of RF radiation, a specific absorption rate (SAR) is determined by taking the volume of the radiation emitted by the antenna array, which can include the surface array of the antenna array and the penetration depth of the RF radiation, the transmission power, which can be averaged based on the dwell time at each frequency during the frequency sweeps to provide a single power measurement, the mass of the tissue irradiated by the RF radiation, and the time average of the delivered power. In order to provide a conservative estimate to provide the maximum radiation emission determination, one or more of the following assumptions can also be made:

While RF can radiate in a cone or other non-prismatic shape, the RF radiation is maximally estimated to be the surface area of the antenna array to determine maximal radiation exposure rates.

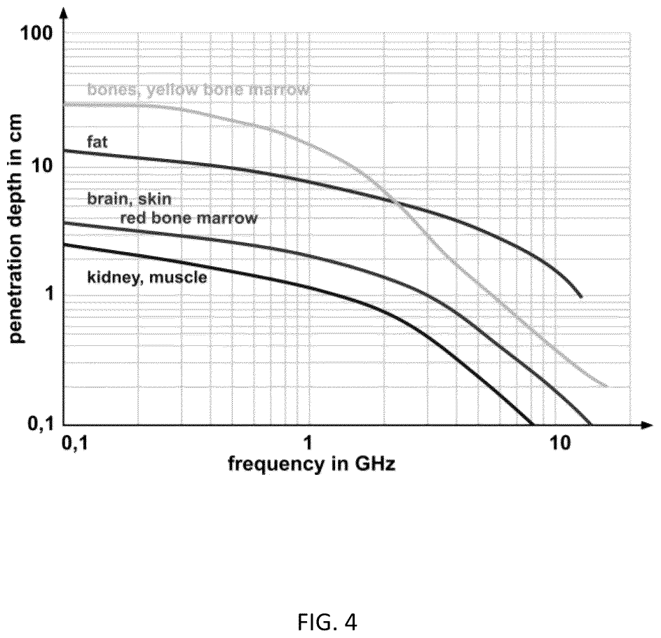

As seen in FIG. 4, while the penetration depth of RF fields in body tissues depends on the frequency and tissue type, e.g., the penetration is lower the higher the frequency, the maximal radiation exposure rate would be for muscle tissue, since the RF radiation at 2.5 GHz has the lowest penetration depth at 0.6 cm, e.g., skin has a penetration depth of approximately 1.2 cm and fat has a penetration depth of approximately 5 cm.

The mass of adipose tissue is used to determine the mass of the tissue irradiated by the analyte sensor, since the mass of skin is 1.090 g/cm³, blood is 1.060 g/cm³, muscle tissue is 1.050 g/cm³, and adipose tissue is 0.950 g/cm³, which that the density of the adipose would give the most radiation emission determination.

The time-average of the system can be based on the measurement taking up to 120 seconds (or 2 minutes) to complete and one measurement taken every 5 minutes.

In view of the above, in an embodiment, in which an analyte sensor has an antenna array having a size of 30×30 mm and transmission power of 16 mW, as measured at the antenna feedpoint, e.g., by a power measurement circuit including, but not limited to, log amplifier(s), diode(s), thermal sensor(s), amplifier(s), transdiode(s), transducer(s), or the like, the SAR can be determined as follows.

Volume of Tissue Exposed to the RF Radiation from a 30×30 mm Antenna Array:

$$3 cm \times 3 cm \times 0.6 cm = 5.4 {cm}^{3}$$

### Mass of Tissue Exposed to the RF Radiation:

$$5.4 {cm}^{3} \times 0.95 g / {cm}^{3} = 5.13 grams$$

### Available Power by Volume:

$$16 mW / 5.13 g = 3.1 mW / g = 3.1 W / kg$$

### Time-Average Power:

$$( 3.1 + 3.1 + 0 + 0 + 0 ) / 5 = 1.24 W / kg .$$

In fact, as seen in Table 1 below, in which a SAR testing of the analyte sensor was performed to determine the SAR level for specific frequencies over 1 gram of tissue, the actual SAR was lower than the conservative estimation of the SAR discussed above, e.g., during a continuous, non-duty cycled operation.

TABLE 1
MEASUREMENT RESULTS

| Frequency | | | Test | Duty | SAR (1 g) | SAR (10 g) | Plot |
|---|---|---|---|---|---|---|---|
| MHz | Side | Spacing | Config | Cycle | (W/kg) | (W/kg) | # |
| 500 | Front | 0 mm | Max Power CW | 1:1 | 0.046 | 0.007 | 1 |
| 650 | Front | 0 mm | Max Power CW | 1:1 | 0.042 | 0.006 | 2 |
| 850 | Front | 0 mm | Max Power CW | 1:1 | 0.083 | 0.014 | 3 |
| 920 | Front | 0 mm | Max Power CW | 1:1 | 0.059 | 0.011 | 4 |
| 1900 | Front | 0 mm | Max Power CW | 1:1 | 0.042 | 0.010 | 5 |
| 2300 | Front | 0 mm | Max Power CW | 1:1 | 0.106 | 0.027 | 6 |
| 2450 | Front | 0 mm | Max Power CW | 1:1 | 0.065 | 0.016 | 7 |
| 3300 | Front | 0 mm | Max Power CW | 1:1 | 0.397 | 0.093 | 8 |
| 3700 | Front | 0 mm | Max Power CW | 1:1 | 0.472 | 0.126 | 9 |
| 4100 | Front | 0 mm | Max Power CW | 1:1 | 0.278 | 0.058 | 10 |

That is, the highest actual SAR level in the frequency range, e.g., 0.106 W/kg, that was found during the testing for the analyte sensor was found to be an order of magnitude below the accepted peak spatial-average SAR level for the user of 1.6 W/kg pursuant to 47 C.F.R. 1.1310.

In fact, a more precise analysis of the SAR that splits the frequencies of the sweep into smaller intervals, and estimates the SAR levels at each smaller interval, suggests that the average SAR over a seven-second period (six seconds of scan plus a one-second pause) is around 0.07 W/kg, as seen in Table 2 below.

TABLE 2

| | | Max SAR in range | Percent | | | | |
|---|---|---|---|---|---|---|---|
| | | (W/kg in 1 gram of | of time | | Time- | | Time- |
| | | tissue); data from | in Range | Time in | weighted | Total SAR | averaged |
| Frequency | Frequency | Table in SAR | (over 6 | range | SAR for | over 6 | SAR |
| start | stop | Report | seconds) | (s) | range (W/kg) | seconds | (W/kg) |
| 500 | 650 | 0.046 | 0.075 | 0.4528 | 0.0208 | 0.4957 | 0.0708 |
| 651 | 850 | 0.083 | 0.100 | 0.5997 | 0.0498 | | |
| 851 | 920 | 0.083 | 0.035 | 0.2099 | 0.0174 | | |
| 921 | 1900 | 0.059 | 0.490 | 2.9385 | 0.1734 | | |
| 1901 | 2300 | 0.106 | 0.200 | 1.1994 | 0.1271 | | |
| 2301 | 2450 | 0.106 | 0.075 | 0.4498 | 0.0477 | | |
| 2451 | 2500 | 0.397 | 0.025 | 0.1499 | 0.0595 | | |

As such, as seen in the above SAR determinations, the analyte sensor as described herein is configured to emit a low amount of energy, even at the worst-case frequency of RF radiation that meets established levels for RF radiation transmission into human tissue, e.g., for telecommunications pursuant to 47 C.F.R. 1.1310. It is noted that this comparison is an extremely conservative determination, at least because radiation power available in the tissue will not all be absorbed.

Furthermore, in determining a full estimate of the tissue heating due to RF radiation exposure, since each frequency has the same dwell time, the value of tissue penetration at each dwell frequency and the power per mass can be calculated and averaged. In a conservative estimate of the time averaged RF radiation emission, the dwell time at the worst-case frequency compared to the overall time can be used. For example, in an embodiment, over the test period, the dwell time at each frequency is 50 ms=0.05 s, which can be used in determining the maximum amount of RF radiation exposure.

In view of the above, at least based on the structure of the analyte sensor, the analyte sensor described herein is configured such that the risk related to RF exposure from the analyte sensor presents very low risk of harm to a user, e.g., safe for the general population, at least because the RF radiation emission is lower than the peak spatial-average SAR of 1.6 W/kg for a user of the analyte sensor according to 47 C.F.R. 1.1310.

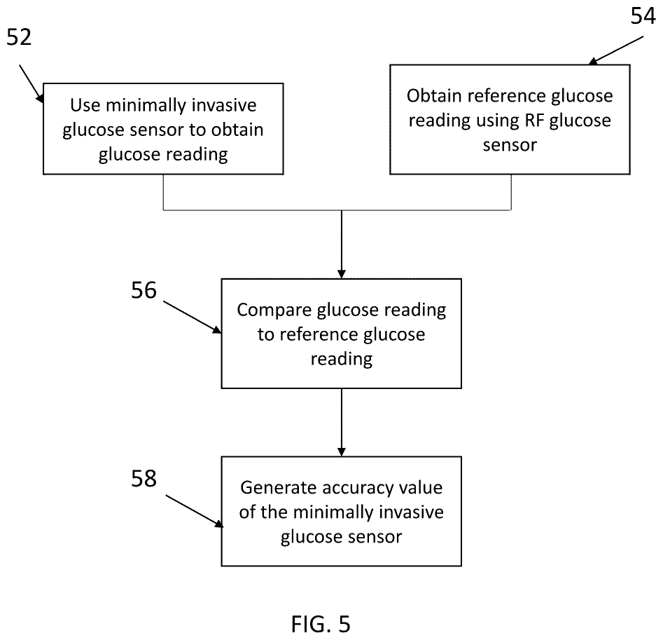

Referring to FIG. 5, an example of using the non-invasive glucose sensor described herein as a reference sensor is illustrated. FIG. 5 schematically depicts a method 50 of using the glucose sensor described herein as a reference or standard glucose sensor against which the accuracy of a minimally invasive glucose sensor, such as a minimally invasive CGM, (or another non-invasive glucose sensor) is determined. At 52, a glucose reading is obtained using the minimally invasive glucose sensor. Contemporaneously, at 54, a reference glucose reading is obtained using the radio/microwave frequency glucose sensor described herein. At 56, the glucose reading from the minimally invasive glucose sensor is compared against the reference glucose reading that is obtained at 54. At 58, an accuracy value of the minimally invasive glucose sensor is then determined based on the comparison at 56.

In an embodiment, the non-invasive glucose sensor as described herein can also be used as a reference to a similar or same non-invasive glucose sensor as described herein. As such, the non-invasive glucose sensors can be used as reference sensors for each other.

It is appreciated that at least in view of the structures of the analyte sensor as described herein, the measurements of the analyte sensor can remain consistent over time while maintaining the accuracy and safety parameters for the analyte sensor.

The analyte sensor described herein can be considered continuous in that it operates substantially continuously to obtain multiple analyte readings over an extended period of time. In another embodiment, the analyte sensor described herein can be considered “on-demand” whereby a user initiates a reading or readings.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A non-invasive glucose sensor that transmits and receives sensing signals in a radio or microwave frequency range of the electromagnetic spectrum, the glucose sensor having at least one antenna that is configured to detect an electromagnetic wave in a radio or microwave frequency range that results from transmission of a transmit signal in the radio or microwave frequency range into a target containing at least glucose, the glucose sensor being configured such that when glucose measurements are obtained from a plurality of adults over a time period that exceeds 1 day, the glucose sensor meets at least one or more of the following:
87% or more of the obtained glucose measurements are within ±20% of corresponding actual glucose values of the plurality of adults;
when the obtained glucose measurements are less than 70 milligrams/deciliter (mg/dL), 85% or more of the obtained glucose measurements are within ±15 mg/dL of the corresponding actual glucose values of the plurality of adults;
when the obtained glucose measurements are between 70 and 180 mg/dL, 70% or more of the obtained glucose measurements are within ±15% of the corresponding actual glucose values of the plurality of adults;
when the obtained glucose measurements are greater than 180 mg/dL, 80% or more of the obtained glucose measurements are within ±15% of the corresponding actual glucose values of the plurality of adults;
when the obtained glucose measurements are less than 70 mg/dL, 98% or more of the obtained glucose measurements are within ±40 mg/dL of the corresponding actual glucose values of the plurality of adults;
when the obtained glucose measurements are between 70 and 180 mg/dL, 99% or more of the obtained glucose measurements are within ±40% of the corresponding actual glucose values of the plurality of adults;
when the obtained glucose measurements are greater than 180 mg/dL, 99% or more of the obtained glucose measurements are within ±40% of the corresponding actual glucose values of the plurality of adults;
when the obtained glucose measurements are less than 70 mg/dL, the corresponding actual glucose values of the plurality of adults are not greater than 180 mg/dL;
when the obtained glucose measurements are greater than 180 mg/dL, the corresponding actual glucose values of the plurality of adults are not less than 70 mg/dL;
when the obtained glucose measurements are less than 70 mg/dL, the corresponding actual glucose values of the plurality of adults are not greater than 180 mg/dL;
when the corresponding actual glucose values of the plurality of adults is less than-2 mg/dL/minute (/min), no more than 1% of the obtained glucose measurements indicate a positive glucose rate of change greater than 1 mg/dL/min; and
when the corresponding actual glucose values of the plurality of adults is greater than 2 mg/dL/min, no more than 1% of the obtained glucose measurements indicate a negative glucose rate of change less than-1 mg/dL/min.

2. The glucose sensor of claim 1, wherein the glucose sensor simultaneously detects glucose from at least two of blood, interstitial fluid, and cellular material.

3. The glucose sensor of claim 1, wherein the glucose sensor is a continuous glucose sensor or an on-demand glucose sensor.

4. The glucose sensor of claim 1, wherein the at least one antenna includes at least two antennas that are part of an antenna array;
a transmit circuit that is electrically connectable to at least one transmit antenna, the transmit circuit configured to generate the signals to be transmitted by the at least one transmit antenna; and
a receive circuit that is electrically connectable to at least one receive antenna, the receive circuit configured to receive responses detected by the at least one receive antenna.

5. The glucose sensor of claim 1, wherein the glucose sensor has from three to six antennas.

6. A non-invasive glucose sensor that transmits and receives sensing signals in a radio or microwave frequency range of the electromagnetic spectrum, the glucose sensor having at least one antenna that is configured to detect an electromagnetic wave in a radio or microwave frequency range that results from transmission of a transmit signal in the radio or microwave frequency range into a target containing at least glucose, the glucose sensor is configured to meet or exceed performance parameters for integrated continuous glucose monitoring systems as set forth in 21 C.F.R. 862.1355 (Feb. 18, 2022).

7. The glucose sensor of claim 6, wherein the glucose sensor simultaneously detects glucose from at least two of blood, interstitial fluid, and cellular material.

8. The glucose sensor of claim 6, wherein the glucose sensor is a continuous glucose sensor or an on-demand glucose sensor.

9. The glucose sensor of claim 6, wherein the at least one antenna includes at least two antennas that are part of an antenna array;
a transmit circuit that is electrically connectable to at least one transmit antenna, the transmit circuit configured to generate the signals to be transmitted by the at least one transmit antenna; and
a receive circuit that is electrically connectable to at least one receive antenna, the receive circuit configured to receive responses detected by the at least one receive antenna.

10. The glucose sensor of claim 6, wherein the glucose sensor has from three to six antennas.

11. An analyte sensor that transmits and receives sensing signals in a radio or microwave frequency range of the electromagnetic spectrum, the analyte sensor having at least one antenna that is configured to detect an electromagnetic wave in a radio or microwave frequency range that results from transmission of a transmit signal in the radio or microwave frequency range into a target containing at least an analyte, the analyte sensor is configured to emit radiofrequency radiation less than radiofrequency radiation exposure limits as set forth in 47 C.F.R. 1.1310 (Apr. 1, 2020).

12. The analyte sensor of claim 11, wherein the analyte sensor is configured to emit the radiofrequency radiation at the peak spatial-average of the SAR at 3.1 W/kg.

13. The analyte sensor of claim 11, wherein the analyte sensor simultaneously detects analyte from at least two of blood, interstitial fluid, and cellular material.

14. The analyte sensor of claim 11, wherein the analyte sensor is a continuous analyte sensor or an on-demand analyte sensor.

15. The analyte sensor of claim 11, wherein the at least one antenna comprises at least two antennas that are part of an antenna array;
a transmit circuit that is electrically connectable to at least one transmit antenna, the transmit circuit configured to generate the signals to be transmitted by the at least one transmit antenna; and
a receive circuit that is electrically connectable to at least one receive antenna, the receive circuit configured to receive responses detected by the at least one receive antenna.

16. The analyte sensor of claim 11, wherein the analyte sensor has from three to six antennas.

17. A analyte sensor that transmits and receives sensing signals in a radio or microwave frequency range of the electromagnetic spectrum, the analyte sensor having at least one antenna that is configured to detect an electromagnetic wave in a radio or microwave frequency range that results from transmission of a transmit signal in the radio or microwave frequency range into a target containing at least an analyte, the analyte sensor is configured to emit radiofrequency radiation according to at least one or more of the following:
a specific absorption rate (SAR) of less than 0.4 W/kg over an entire body of a non-user exposed to the analyte sensor;
a peak spatial-average of the SAR of less than 8 W/kg to the non-user wearing the analyte sensor averaged over any 1 gram of tissue of the non-user;
the SAR of less than 0.08 W/kg for a user of the analyte sensor averaged over an entire body of the user; and
the peak spatial average of the SAR of less than 1.6 W/kg for the user of the analyte sensor averaged over any 1 gram of tissue from the entire body of the user.

18. The analyte sensor of claim 17, wherein the analyte sensor is configured to emit the radiofrequency radiation at the peak spatial-average of the SAR at 3.1 W/kg.

19. The analyte sensor of claim 17, wherein the analyte sensor simultaneously detects analyte from at least two of blood, interstitial fluid, and cellular material.

20. The analyte sensor of claim 17, wherein the analyte sensor is a continuous analyte sensor or an on-demand analyte sensor.

21. The analyte sensor of claim 17, wherein the at least one antenna comprises at least two antennas that are part of an antenna array;
a transmit circuit that is electrically connectable to at least one transmit antenna, the transmit circuit configured to generate the signals to be transmitted by the at least one transmit antenna; and
a receive circuit that is electrically connectable to at least one receive antenna, the receive circuit configured to receive responses detected by the at least one receive antenna.

22. The analyte sensor of claim 17, wherein the analyte sensor has from three to six antennas.
